(19) United States
(12) Patent Application Publication
(10) Pub. No.: US 2014/0276707 A1
(43) Pub. Date: Sep. 18, 2014

(54) SYSTEM AND METHOD OF USING EVOKED COMPOUND ACTION POTENTIALS TO MINIMIZE VESSEL TRAUMA DURING NERVE ABLATION

(71) Applicant: BOSTON SCIENTIFIC NEUROMODULATION CORPORATION, Valencia, CA (US)

(72) Inventor: Kristen Jaax, Santa Clarita, CA (US)

(73) Assignee: BOSTON SCIENTIFIC NEUROMODULATION CORPORATION, Valencia, CA (US)

(21) Appl. No.: 14/201,884

(22) Filed: Mar. 9, 2014

Related U.S. Application Data
(60) Provisional application No. 61/801,354, filed on Mar. 15, 2013.
(60) Provisional application No. 61/808,229, filed on Apr. 4, 2013.

(51) Int. Cl. A61B 18/02; A61B 18/14

(52) U.S. Cl. CPC A61B 18/02; A61B 18/1492
USPC 606/21; 606/31

(57) ABSTRACT

A therapy system for use with a patient and a method of treating a medical condition of a patient. Electrical stimulation energy is delivered to a stimulation site on the wall of a blood vessel, thereby evoking at least one compound action potential in a nerve branch associated with the blood vessel. The evoked compound action potential(s) is sensed at a sensing site on the wall of the blood vessel. A circumferential location of the nerve branch is identified as being adjacent one of the stimulation site and the sensing site based on the sensed compound action potential(s). Ablation energy is delivered to an ablation site on the wall of the blood vessel adjacent the circumferential location of the nerve branch, thereby ablating the nerve branch and treating the medical condition.

### RELATED APPLICATIONS DATA

The present application claims the benefit under 35 U.S.C. §119 to U.S. Provisional Application Ser. No. 61/801,354, filed Mar. 15, 2013 and U.S. Provisional Application Ser. No. 61/808,229, filed Apr. 4, 2013, which applications are all incorporated herein by reference in their entirety.

### FIELD OF INVENTION

The present invention relates to systems for treating hypertension in patients.

### BACKGROUND OF THE INVENTION

Hypertension is a health problem affecting millions of people, requiring considerable expenditure of medical resources as well as imposing significant burdens on those who suffer from this condition. Hypertension generally involves resistance to the free flow of blood within a patient's vasculature, often caused by reduced volume stemming from plaque, lesions, and the like. Because blood vessels do not permit easy flow, the patient's heart must pump at higher pressure. In addition, reduced cross-sectional area results in higher flow velocity. In consequence, a patient's blood pressure may enter into the range of hypertension, i.e. greater than 140 mm Hg systolic/90 mm Hg diastolic.

Certain treatments for congestive heart failure or hypertension require the temporary or permanent interruption or modification of select nerve function in the renal blood vessel. In one scenario, the kidneys produce a sympathetic response to congestive heart failure, which, among other effects, increases the undesired retention of water and/or sodium. Ablating some of the nerves running to the kidneys may reduce or eliminate this sympathetic function, which may provide a corresponding reduction in the associated undesired symptoms

In this process of ablating renal nerves, an ablation element is carried in an instrument such as an endoscope, is introduced into a patient's vasculature and navigated to a position within the renal artery. Ablation energy, such as thermal ablation energy or cyroablation energy, is applied to the ablative element, resulting in the destruction of the renal nerves. Although this process is effective in combating hypertension, the conventional renal nerve ablation methods ablate tissue in a circumferential pattern within the renal artery with no knowledge of the specific locations of the target renal nerve branches, thereby causing unnecessary weakening of the vessel wall.

Thus, there exists a need for a better procedure that can treat hypertension with focused ablation of targeted nerves.

### SUMMARY OF THE INVENTION

In accordance with a first aspect of the present inventions, a therapy system for use with a patient is provided. The therapy system comprises a cylindrical support structure configured for being deployed in a blood vessel of the patient. The cylindrical support structure carries a plurality of electrodes circumferentially disposed about the cylindrical support structure (e.g., a stent or a balloon), and a plurality of ablative elements circumferentially disposed about the cylindrical support structure respectively adjacent the plurality of electrodes. The ablative elements may comprise the electrodes. In one embodiment, the cylindrical support structure comprises a resilient skeletal spring structure for urging the plurality of electrodes and plurality of ablative elements against an inner wall of the blood vessel. The cylindrical support structure may comprise an electrically insulative material for preventing electrical energy from being radially conveyed inward from the cylindrical support structure. The therapy system further comprises an electrode configured for being deployed in the blood vessel of the patient at a location axially remote from the plurality of electrodes. The electrode may be a ring electrode and may be carried by the cylindrical support structure.

The therapy system further comprises stimulation output circuitry, monitoring circuitry, and a controller/processor configured for performing at least one of a first process and a second process. The first process comprises prompting the stimulation output circuitry to sequentially activate the plurality of electrodes to evoke at least one compound action potential (CAP) in a nerve associated with the blood vessel, prompting the monitoring circuitry to activate the axially remote electrode in response to the activation of each of the plurality of electrodes to sense the evoked CAP(s) (eCAPs), and identifying one of the plurality of electrodes based on the sensed eCAP(s). The second process comprises prompting the stimulation output circuitry to active the axially remote electrode to evoke at least one CAP in the nerve associated with the blood vessel, prompting the monitoring circuitry to sequentially activate the plurality of electrodes in response to the activation of the axially remote electrode to sense the eCAP(s), and identifying the one electrode based on the sensed eCAP(s). In an optional embodiment, a plurality of CAPs are evoked and sensed to increase the signal-to-noise ratio of the sensed eCAPs.

The therapy system further comprises an ablation source (e.g., a thermal ablation source or a cryoablation source) configured for delivering ablation energy to ablative element adjacent the identified electrode.

In accordance with a second aspect of the present inventions, a method for treating a medical condition (e.g., hypertension) of a patient will be provided. The method comprises delivering electrical stimulation energy to a stimulation site on the wall of a blood vessel (e.g., a renal artery), thereby evoking at least one CAP in a nerve branch associated with the blood vessel. The method further comprises sensing the eCAP(s) at a sensing site on the wall of the blood vessel. In an optional method, a plurality of CAPs are evoked and sensed to increase signal-to-noise ratio of the sensed eCAPs. The method may optionally further comprise disposing a stimulating electrode in the blood vessel at the stimulation site, in which case, the electrical stimulation energy is delivered by the stimulating electrode, and disposing a sensing electrode in the blood vessel at the sensing site, in which case, the eCAP(s) is sensed by the sensing electrode.

The method further comprises identifying a circumferential location of the nerve branch as being adjacent one of the stimulation site and the sensing site based on the sensed eCAP. The method further comprises delivering ablation energy (e.g., thermal ablation energy or cryoablation energy) to an ablation site on the wall of the blood vessel adjacent the circumferential location of the nerve branch, thereby ablating the nerve branch and treating the medical condition. In this case where hypertension is treated, and the blood vessel is a renal artery, the ablation of the nerve branch may decrease the blood pressure of the patient, thereby treating the hypertension.

In the case where the identified circumferential location of the nerve branch is adjacent the stimulation site, the method may further comprise disposing a plurality of stimulation electrodes in the blood vessel respectively at a plurality of circumferential sites in axial alignment with the stimulation site, disposing a sensing electrode in the blood vessel at the sensing site, and sequentially activating the stimulation electrodes, one of which will evoke the CAP(s). The method further comprises activating the sensing electrode in response to the activation of each of the stimulation electrodes to sense the eCAP(s), and identifying the circumferential site at which the one stimulation electrode is located as the stimulation site. The method may further comprise disposing a plurality of ablative elements in the blood vessel respectively adjacent the stimulation electrodes (the ablative elements may simply comprise the stimulation electrodes), and selecting the ablative element adjacent the one stimulation electrode to convey the ablation energy to the ablation site. The method may further comprise disposing a cylindrical support structure in the blood vessel in axial alignment with the stimulation site. In this case, the stimulation electrodes and ablative elements are carried by the cylindrical support structure.

In the case where the identified circumferential location of the nerve branch is adjacent the sensing site, the method may further comprise disposing a plurality of sensing electrodes in the blood vessel respectively at a plurality of circumferential sites in axial alignment with the sensing site, disposing a stimulation electrode in the blood vessel at the stimulation site, activating the stimulation electrode to evoke the CAP(s), and sequentially activating the sensing electrodes in response to the activation of the stimulation electrode. The eCAP(s) may be sensed by the activation of one of the sensing electrodes. The method further comprises identifying the circumferential site at which the one sensing electrode is located as the sensing site. The method may further comprise disposing a plurality of ablative elements in the blood vessel respectively adjacent the sensing electrodes (the ablative elements may simply comprise the sensing electrodes), and selecting the ablative element adjacent the one sensing electrode to convey the ablation energy to the ablation site. The method further comprises disposing a cylindrical support structure in the blood vessel in axial alignment with the sensing site. In this case, the sensing electrodes and ablative elements are carried by the cylindrical support structure.

Other and further aspects and features of the invention will be evident from reading the following detailed description of the preferred embodiments, which are intended to illustrate, not limit, the invention.

### BRIEF DESCRIPTION OF DRAWINGS

The drawings illustrate the design and utility of preferred embodiments of the present invention, in which similar elements are referred to by common reference numerals. In order to better appreciate how the above-recited and other advantages and objects of the present inventions are obtained, a more particular description of the present inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is block diagram of a nerve ablation system arranged in accordance with one embodiment of the present inventions, wherein the nerve ablation system is shown in use with a patient suffering from hypertension;

FIG. 2 is a profile view of an exemplary stent lead used in the nerve ablation system of FIG. 1;

FIG. 3 is a flow diagram illustrating one method of using the nerve ablation system of FIG. 1 to identify and ablate renal nerve branches, thereby treating the hypertension of the patient;

FIG. 4 is a flow diagram illustrating another method of using the nerve ablation system of FIG. 1 to identify and ablate renal nerve branches, thereby treating the hypertension of the patient; and

FIG. 5 is a perspective view illustrating the stent lead of the nerve ablation system of FIG. 1 deployed within a renal artery of the patient.

### DETAILED DESCRIPTION OF THE EMBODIMENTS

While the devices and methods described herein are discussed relative to renal nerve modulation, it is contemplated that the devices and methods may be used in other locations and/or applications where nerve modulation and/or other tissue modulation including heating, activation, blocking, disrupting, or ablation are desired, such as, but not limited to: blood vessels, urinary vessels, or in other tissues via trocar and cannula access. For example, the devices and methods described herein can be applied to nerve excitation or blocking or ablation, modulation of muscle activity, hyperthermia or other warming of tissues, etc. In some instances, it may be desirable to ablate perivascular renal nerves with ultrasound ablation. The term ablation refers to techniques that may permanently alter the function of nerves and other tissue such as brain tissue or cardiac tissue. When multiple ablations are desirable, they may be performed sequentially by a single ablation device.

Turning first to FIG. 1, an exemplary nerve ablation system 10 constructed in accordance with one embodiment of the present inventions will be described. The system 10 generally comprises a stent catheter 12, a guide sheath 16, an external control unit 18 and return electrode patches 20A and 20B, (collectively, electrode patches 20). The proximal end of the stent catheter 12 is connected to the control unit 18, which supplies the necessary stimulation and/or ablation energy to activate the stent catheter 12. The return electrode patches 20 are connected to the control unit 18 and may be attached on the patient's skin, such as the legs and/or at another conventional location on the patient's body, to complete the circuit.

As shown in FIG. 2, the stent catheter 12 comprises an elongated catheter body 22, a cylindrical support structure 24 configured for being deployed in a blood vessel of the patient, and a plurality of electrodes 26 carried by the cylindrical support structure 24. The electrodes 26, which may function as stimulation electrodes, ablation electrodes, and/or sensing electrodes, are circumferentially and axially disposed about the cylindrical support structure 24. By way of non-limiting example, the cylindrical support structure 24 carries twenty-four electrodes 26, arranged as three rings of electrodes axially located relative to each other (the first ring A consisting of electrodes E1-E8; the second ring B consisting of electrodes E8-E16; and the third ring C consisting of electrodes E17-E24). The actual number of electrodes will, of course, vary according to the intended application.

In one embodiment, each of the electrodes 26 may be configured as either a stimulation electrode, a sensing electrode, or an ablation electrode. In another embodiment, all of the electrodes located on a ring, such as the ring A, are configurable as stimulation electrodes, and all of the electrodes located on a separate ring, such as the ring C, are configurable as sensing electrodes. Some of the stimulation electrodes or sensing electrodes may be reconfigured as ablation electrodes.

Alternatively, some of the electrodes 26 may be dedicated ablation electrodes. For example, the odd-numbered electrodes on the first ring A may be dedicated stimulation electrodes, the odd-numbered electrodes on the third ring C may be dedicated sensing electrodes, the odd numbered electrodes on the second ring B may be either dedicated stimulation electrodes or dedicated sensing electrodes. The even-numbered electrodes spread across all the rings A, B, C may be dedicated ablation electrodes.

In alternative embodiments where the ablative elements are not electrodes, none of the electrodes on rings A, B, C are ablation electrodes. In this case, the ablative elements can be circumferentially arranged around the cylindrical support structure 24 in proximity to the stimulation electrodes or sensing electrodes. Although the electrode rings A, B, C are illustrated as being carried by a single stent catheter for purposes of convenience, at least two of the electrode rings A, B, C can be located on separate stent catheters. In some other embodiments, rather than using one or more of the electrodes 26 on the rings A, B, and C as ablation electrodes, the stent catheter 12 may include a movable or adjustable roving ablation element (not shown) that may be located at one of the sites adjacent the stimulation electrodes and/or sensing electrodes. In any event, the significance is that there be one or more ablation elements that are located or locatable about the circumference of the cylindrical support structure 24.

The cylindrical support structure 24 takes the form of a resilient skeletal spring structure that allows it to be collapsed into low-profile geometry to facilitate convenient delivery of the stent catheter 12 into the blood vessel, and spring open or expand for urging the electrodes 26 against an inner wall of the blood vessel. The resilient skeletal spring structure 24 may be made from a wire having a relatively high-stiffness and resilient material or a high-stiffness urethane or silicone, that is shaped into a three-dimensional geometry. In an alternative embodiment, the cylindrical support structure 24 takes the form of a balloon that can expand from a low-profile geometry to an expanded geometry.

The electrodes 26 are disposed on the outer surface of the cylindrical support structure 24. In this setting, when the cylindrical support structure 24 is expanded within the blood vessel, all the electrodes 26 are arranged to point outward from the cylindrical support structure 24 and deliver stimulation energy to the vessel wall (in order to evoke compound action potentials (CAPs) in nerve branches associated with the vessel as will be described in further detail below), sense physiological information from the vessel wall (in order to sense the evoked CAPs (eCAPs) from the nerve branches associated with the vessel as will be described in further detail below), and/or deliver ablation energy to the vessel wall (in order to ablate the nerve branches associated with the vessel as will be described in further detail below). The regions where the electrodes 26 configured as the stimulation electrodes, the sensing electrodes, and the ablation electrodes come in contact with the inner wall of the blood vessel are called as stimulation sites, sensing sites, and ablation sites, respectively.

The stent catheter 12 further comprises an electrical insulation structure 28 disposed on the luminal surface of the cylindrical support structure 24 for preventing the electrical energy or the ablation energy from being radially conveyed inward from the electrodes 26 to the blood and for preventing physiological information from being sensed from the blood. The electrical insulation structure 28 may be made of a flexible electrical insulation layer formed of a relatively thin (e.g., 0.1 mm to 2 mm, although 1 mm or less is most preferred) and relatively low-stiffness material. Exemplary materials are low-stiffness silicone, expanded polytetrafluorethylene (ePTFE), or urethane. Further details describing the construction and method of manufacturing stent lead are disclosed in U.S. Patent Publication. No. 2012/0059446 A1, entitled “Collapsible/Expandable Tubular Electrode Leads,” which is expressly incorporated herein by reference.

The control unit 18 is configured for delivering electrical stimulation energy in the form of a pulsed electrical waveform (i.e., a temporal series of electrical pulses) to the stimulation electrodes, thereby evoking compound action potentials (eCAPs) within nerves, sensing the eCAPs at the sensing electrodes, and delivering ablation energy to the ablation electrodes. As will be described in detail later below, the system 10 identifies the electrodes that are adjacent (or sufficiently close) to the nerve branches based on the eCAP measurements. The system 10 then uses those identified electrodes as reference points to deliver ablation energy to adjacent nerve branches.

The control unit 18 comprises a controller/processor 30, stimulation output circuitry 32, monitoring circuitry 34, an ablation source 36, and other suitable components (not shown) known to those skilled in the art. The controller/processor 30 executes a suitable program stored in a memory (not shown) for controlling the stimulation output circuitry 32 and monitoring circuitry 34 to evoke and sense eCAPs in nerve branches associated with the blood vessel, identifying target sites on the nerve branches based on the sensed eCAPs, and controlling the ablation source 36 to ablate the identified target sites. In performing these functions, the controller/processor 30 configures (to the extent that the electrodes 26 are reconfigurable) selected ones of the electrodes 26 as stimulation electrodes, sensing electrodes, and ablation electrodes at the appropriate times.

The modulation output circuitry 32 is configured for delivering electrical stimulation energy in the form of a pulsed electrical waveform to the electrodes 26 activated as stimulation electrodes in accordance with a set of stimulation parameters. The stimulation parameter set includes an electrode combination parameter for defining the electrodes 26 to be activated as anodes (positive), cathodes (negative) and turned off (zero). The stimulation parameter set further includes an electrical pulse parameter, which defines the pulse amplitude (measured in milliamps or volts depending on whether the control block 18 supplies constant current or constant voltage to the electrodes 26), pulse width (measured in microseconds), and pulse rate (measured in pulses per second) of the electrical stimulation energy.

With respect to the delivery of stimulation energy, electrodes that are selected to transmit or receive electrical energy are referred to herein as “activated,” while electrodes that are not selected to transmit or receive electrical energy are referred to herein as “non-activated.” Electrical energy delivery will occur between two (or more) electrodes, one of which may be the patch electrodes 20, so that the electrical current has a path from the stimulation output circuitry 32 to the tissue and a sink path from the tissue to the stimulation output circuitry 32. Electrical energy may be transmitted to the tissue in a monopolar or multipolar (e.g., bipolar, tripolar, etc.) fashion, or by any other means available.

Monopolar delivery occurs when a selected one or more of the stent catheter electrodes 26 is activated along with the patch electrodes 20, so that electrical energy is transmitted between the selected electrodes 26 and the patch electrodes 20. Monopolar delivery may also occur when one or more of the electrodes 26 are activated along with a large group of lead electrodes (which may include the patch electrodes 22) located remotely from the stent catheter electrode(s) 26 so as to create a monopolar effect; that is, electrical energy is conveyed from the stent catheter electrode(s) 26 in a relatively isotropic manner. Bipolar delivery occurs when two of the stent catheter electrodes 26 are activated as anode and cathode, so that electrical energy is transmitted between the stent catheter electrodes 26. Tripolar delivery occurs when three of the stent catheter electrodes 26 are activated, two as anodes and the remaining one as a cathode, or two as cathodes and the remaining one as an anode.

The monitoring circuitry 34 is configured for monitoring status of various nodes and parameters throughout the control unit 18, e.g., power supply voltages, temperature, and the like. More significantly, the monitoring circuitry 32 is configured for sensing eCAPs at the sensing electrodes 26. The ablation source 36 is configured for delivering ablation energy to the ablation electrodes 26. In the illustrated embodiment, the ablation source 36 is a radio frequency (RF) source. Alternatively, ablation sources, such as, ultrasound, laser, or cryoablation energy sources may be used. In these alternative embodiments, ablation elements other than electrodes may be located on the stent catheter 12.

The nerve ablation system 10 (shown in FIG. 1) may be employed to identify ablation sites adjacent the renal nerve branches and ablate these sites to treat hypertension. Specifically, the nerve ablation system 10 is configured for delivering modulation energy to the renal artery for identifying the location of renal nerve branches and delivering ablation energy to the renal nerve branches in an attempt to disrupt the renal nerve branches, thereby affecting the patient's blood pressure. For example, ablation of the renal nerve branches that form a portion of the sympathetic nervous system, may reduce sympathetic tone, which in turn has an electrical sympatholytic effect, producing a reduction in the patient's blood pressure. That is, ablation of the renal nerve branches may block action potentials that down-regulate the sympathetic nervous system, resulting in vasodilation, thus decreasing the patient's blood pressure.

To this end, the controller/processor 30 is configured for performing at least one of two techniques for identifying a renal nerve to be ablated.

In the first technique, the controller/processor 30 prompts the stimulation output circuitry 32 to sequentially activate the stimulation electrodes located on one of the rings (A, B, or C) to evoke at least one CAP in one of the renal nerve branches. The controller/processor 30 prompts the monitoring circuitry 32 to simultaneously activate the sensing electrodes located on a different one of the rings (A, B, or C) (or alternatively, a single ring electrode (not shown)) in response to the sequential activation of each of the stimulation electrodes.

At least one of the sensing electrode(s) senses the eCAP(s), and based on this sensing, the controller/processor 30 identifies at least one of the stimulation electrodes located adjacent to the nerve branch. That is, the stimulation electrode that evoked the CAP that was sensed by one of the sensing electrodes will be identified as the electrode that is adjacent the nerve branch. To increase the signal-to-noise ratio, the multiple CAPs may be evoked by each stimulation electrode and sensed by the sensing electrode(s). The controller/processor 30 may then average the magnitudes of multiple CAPs evoked by each stimulation electrode, and then use this average to identify the stimulation electrode(s) that are located adjacent to the nerve branch.

The controller/processor 30 prompts the ablation source 36 to deliver ablation energy to the ablation electrode adjacent the identified stimulation electrode, thereby ablating the nerve branch. The ablation electrode may be the identified stimulation electrode, one of the electrodes adjacent the identified stimulation electrode, or even two electrodes circumferentially flanking the identified stimulation electrode. In the latter case, the two electrodes may be operated in a bipolar manner to ablate the tissue, including the nerve branch, located between the two ablation electrodes.

In the second technique, the controller/processor 30 prompts the stimulation output circuitry 32 to simultaneously activate the stimulation electrodes located on one of the rings (A, B, or C) (or alternatively, a single ring electrode (not shown) to evoke at least one CAP in one of the renal nerve branches. The controller/processor 30 prompts the monitoring circuitry 34 to sequentially activate the sensing electrodes located on a different one of the rings (A, B, or C) in response to the simultaneous activation of the stimulation electrodes.

At least one of the sensing electrode(s) senses the eCAP(s), and based on this sensing, the controller/processor 30 identifies at least one of the sensing electrodes located adjacent to the nerve branch. That is, the sensing electrode that sensed the eCAP that was evoked by one of the stimulation electrodes will be identified as the electrode that is adjacent the nerve branch. To increase the signal-to-noise ratio, the multiple eCAPs may be sensed by each of the sensing electrodes. The controller/processor 30 may then average the magnitudes of the multiple CAPs sensed by each sensing electrode, and then use this average to identify the sensing electrode(s) that are located adjacent to the nerve branch.

The controller/processor 30 prompts the ablation source 36 to deliver ablation energy to the ablation electrode adjacent the identified sensing electrode, thereby ablating the nerve branch. The ablation electrode may be the identified sensing electrode, one of the electrodes adjacent the identified sensing electrode, or even two electrodes circumferentially flanking the identified sensing electrode. In the latter case, the two electrodes may be operated in a bipolar manner to ablate the tissue, including the nerve branch, located between the two ablation electrodes.

Having described the structure and function of the nerve ablation system 10, one method 100 of using the system 10 to treat hypertension in a patient will now be described with reference to FIG. 3. Although this method is described in the context of treating hypertension, it should be appreciated that the method can be modified to treat various other medical conditions, such as those pertaining pulmonary and cardiac diseases.

First, the support structure 24 of the stent catheter 12 is deployed in the renal artery in a conventional manner (step 102). In particular, the support structure 24, while in the collapsed state, is advanced through the guide sheath 16 and placed into the renal artery, as shown in FIG. 1. As the support structure 24 is advanced from the distal end of the guide sheath 16, it expands to firmly place the electrodes 26 against the inner wall of the blood vessel. Thus, the three electrode rings (A, B, and C) are disposed in the renal artery, as illustrated in FIG. 5. In this example, the electrode ring A will be used to evoke the CAPs, whereas the electrode ring C will be used to sense the eCAPs. In this case, electrodes E1-E8 will be disposed at a plurality of circumferential stimulation sites within the renal artery, and electrodes E17-E24 will be disposed at a plurality of sensing sites within the renal artery axially remote from the circumferentially disposed stimulation sites. Alternatively, if a single sensing ring electrode is used, it will be disposed at a single circumferential sensing site axially remote from the circumferentially disposed stimulation sites. In this technique, the ablation electrodes will be disposed in axial alignment with the circumferentially disposed stimulation sites within the renal artery. In the illustrated method, the ablation electrodes are identical to the ring of stimulation electrodes E1-E8, and thus, the stimulation sites are equivalent to the ablation sites. However, as previously discussed above, the ring of electrodes may alternate between stimulation electrodes and ablation electrodes (e.g., stimulation electrodes being electrodes E1, E3, E5, and E7; and ablation electrodes being electrodes E2, E4, E6, and E8), in which case, the ablation sites and the stimulation sites will alternate between each other.

It is contemplated that at least one of the stimulation electrodes and at least one of the sensing electrodes will be located adjacent to a nerve branch within the wall of the blood vessel. In the example illustrated in FIG. 5, one nerve branch (nerve branch 1) extends along the renal artery in proximity to stimulation electrode E2 and sensing electrode E18, and another nerve branch (nerve branch 2) extends along the renal artery in proximity to stimulation electrode E7 and sensing electrode E23. It should be noted the stimulation electrode and sensing electrode that are adjacent a particular renal nerve branch may not be on the same circumferential location. For example, stimulation electrode E7 and sensing electrode E23 are circumferentially offset from each other by one electrode.

Next, the controller/processor 30 prompts the modulation output circuitry 32 to sequentially activate the stimulation electrodes one-at-a-time to deliver the electrical stimulation energy to the wall of the renal artery at the respective stimulation sites (step 104). If any nerve branch is present at any of the stimulation sites, the stimulation energy depolarizes that nerve branch, thereby evoking a CAP that propagates along the nerve branch. For example, delivering the electrical stimulation energy from electrodes E2 and E7 should respectively evoke CAPs in nerve branches 1 and 2. Such stimulation is supra-threshold, but should not be uncomfortable for a patient. A suitable stimulation pulse is, for example, 4 mA for 200 μs.

The controller/processor 30 optionally prompts the stimulation output circuitry 32 to activate each stimulation electrode multiple times to deliver the electrical stimulation energy to the wall of the renal artery at each stimulation site. In this case, each stimulation electrode may be activated multiple times without any intervening activation of other stimulation electrodes, or the stimulation electrodes may be cyclically activated multiple times. In either event, each stimulation electrode may be activated multiple times. If the nerve branch is present at any stimulation site, multiple CAPs will be evoked at this stimulation site.

In response to the activation of each stimulation electrode, the controller/processor 30 prompts the monitoring circuitry 34 to simultaneously activate the sensing electrodes (or alternatively, activated a ring electrode) to sense the eCAP(s) at the sensing site(s) (step 106). In the case where the stimulation electrodes are activated multiple times to evoke multiple eCAP(s) in the nerve branches for each sensing electrode, the multiple eCAPs that are sensed will be averaged to increase the signal-to-noise ratio of all eCAPs sensed by the sensing electrodes.

In the illustrated example, stimulation electrode E1 will be activated, but will not evoke an eCAP, since it is not adjacent any of nerve branches 1 and 2. In response, the sensing electrodes E17-E24 will be activated, but will not sense an eCAP since none has been evoked. Stimulation electrode E2 will then be activated, and will evoke an eCAP, since it is adjacent nerve branch 1. In response, the sensing electrodes E17-E24 will be activated, and will sense the eCAP. This process is repeated for each of remaining electrodes E3-E8, with electrodes E3-E6 and E8 not evoking an eCAP, since none are adjacent the any of nerve branches 1 and 2, and electrode E7 will evoke an eCAP, since it is adjacent nerve branch 2. It can be determined from this that electrodes E2 and E7 are respectively adjacent nerve branches 1 and 2.

Next, the controller/processor 30 identifies the stimulation electrode that evoked the CAP, and thus, the circumferential location of the nerve branch (step 108). That is, the stimulation electrode that evoked the CAP that was sensed by any of the sensing electrodes will be deemed the stimulation electrode that is adjacent the nerve branch. In the illustrated embodiment, electrodes E2 and E7 will be identified as the stimulation electrodes that are adjacent respective nerve branches 1 and 2.

Then, the controller/processor 30 prompts the ablation source 36 to deliver ablation energy to the ablation electrode(s) adjacent the identified stimulation site(s) (i.e., the stimulation site(s) that are adjacent the renal nerve branch(es)) (step 110). As previously discussed above, the ablation electrode may be any of the electrodes E1-E8, and in this case, electrodes E2 and E7, which may be activated in a monopolar manner in conjunction with the patch electrodes to ablate nerve branches 1 and 2. In the case where only odd electrodes are used as stimulation electrodes, and even electrodes are used as ablation electrodes, the stimulation electrodes that may be identified as being adjacent to the nerve branches may be electrodes E3 and E7. In this case, electrodes E2 and E4 may be activated in a bipolar manner to ablate nerve branch 1, and electrodes E6 and E8 can be activated in a bipolar manner to ablate nerve branch 2. As a result of the ablation of the renal nerve branch(es), the blood pressure of the patient will be lowered, thereby treating the hypertension.

The ablation energy may be delivered under any one of the two approaches. In a first approach, the ablation energy is delivered to the site(s) of the nerve branch(es) at an intensity that ensures that the nerve branch(es) is completely ablated. In a second approach, ablation energy of relatively lesser intensity may be delivered to the site(s) of the nerve branch(es) to create a relatively smaller ablation for minimizing any unintended vessel wall damage. Subsequently, mapping of the viable nerve branch(s) is performed, as discussed in steps 104, 106, and 108 to determine whether the nerve branch(es) were successfully ablated. If not, the lesion may be expanded by redelivering the ablation energy to the site(s) of the nerve branch(es). This process is iteratively repeated until the nerve branch(s) are completely ablated. Seconds, minutes, or months may elapse between ablations.

Another method 200 of using the system 10 to treat hypertension in a patient will now be described with reference to FIG. 4. The method 200 is similar to the method 100 with the exception that the ablation energy is delivered to sensing sites that are adjacent to the renal nerve branches.

First, the support structure 24 of the stent catheter 12 is deployed in the renal artery in the same manner described above with respect to step 102 (step 202). Next, the controller/processor 30 prompts the modulation output circuitry 32 to simultaneously activate the stimulation electrodes (or alternatively, a single ring electrode) multiple times to deliver the electrical stimulation energy to the wall of the renal artery at the respective stimulation sites (step 204). The stimulation energy depolarizes the nerve branches, thereby evoking CAPs that propagate along each nerve branch. In response to the activation of the stimulation electrodes, the controller/processor 30 prompts the monitoring circuitry 34 to sequentially activate the sensing electrodes (to sense the eCAPs at the sensing sites (step 206). That is, each time the stimulation electrodes are simultaneously activated, a different one of the sensing electrodes is activated.

For each sensing electrode, the controller/processor 30 optionally prompts the stimulation output circuitry 32 to activate the stimulation electrodes multiple times to deliver the electrical stimulation energy to the wall of the renal artery at the stimulation sites, thereby evoking multiple CAPs at each of the renal nerve branches. The multiple eCAPs that are sensed will be averaged to increase the signal-to-noise ratio of all eCAPs sensed by each sensing electrode.

In the illustrated example, stimulation electrodes E1-E8 will be activated to evoke eCAPs in nerve branches 1 and 2. In response, sensing electrode E17 may be activated, but will not sense the evoked eCAPs, since it is not adjacent nerve branches 1 and 2. Stimulation electrodes E1-E8 will be activated again to evoke eCAPs in nerve branches 1 and 2. In response, sensing electrode E18 may be activated, and will sense an evoked eCAP, since it is adjacent nerve branch 1. This process is repeated for each of remaining sensing electrodes E19-E24, with sensing electrodes E19-E21, and E23-E24 not sensing an eCAP, since that are not adjacent nerve branches, and sensing electrode E22 sensing an eCAP, since it is adjacent nerve branch 2. It can be determined from this that electrodes E18 and E22 are respectively adjacent nerve branches 1 and 2.

Next, the controller/processor 30 identifies the sensing electrode that sensed the CAP, and thus, the circumferential location of the nerve branch (step 208). That is, the sensing electrode that sensed the CAP that was evoked by any of the stimulation electrodes will be deemed the sensing electrode that is adjacent the nerve branch. In the illustrated embodiment, electrodes E18 and E22 will be identified as the sensing electrodes that are adjacent respective nerve branches 1 and 2.

Then, the controller/processor 30 prompts the ablation source 36 to deliver ablation energy to the ablation electrode(s) adjacent the identified sensing site(s) (i.e., the sensing site(s) that are adjacent the renal nerve branch(es)) (step 210). As previously discussed above, the ablation electrode may be any of the electrodes E17-E24, and in this case, electrodes E18 and E22, which may be activated in a monopolar manner in conjunction with the patch electrodes to ablate nerve branches 1 and 2. In the case where only odd electrodes are used as stimulation electrodes, and even electrodes are used as ablation electrodes, the sensing electrodes that may be identified as being adjacent to the nerve branches may be electrodes E19 and E23. In this case, electrodes E18 and E20 may be activated in a bipolar manner to ablate nerve branch 1, and electrodes E22 and E24 can be activated in a bipolar manner to ablate nerve branch 2. As a result of the ablation of the renal nerve branch(es), the blood pressure of the patient will be lowered, thereby treating the hypertension. The ablation energy may be delivered in accordance with any one of the two approaches described above.

Although particular embodiments of the present inventions have been shown and described, it will be understood that it is not intended to limit the present inventions to the preferred embodiments, and it will be obvious to those skilled in the art that various changes and modifications may be made without departing from the spirit and scope of the present disclosure. Thus, the present inventions are intended to cover alternatives, modifications, and equivalents, which may be included within the spirit and scope of the present inventions as defined by the claims.

What is claimed is:

1. A therapy system for use with a patient, comprising:
a cylindrical support structure configured for being deployed in a blood vessel of the patient;
a plurality of electrodes circumferentially disposed about the cylindrical support structure;
a plurality of ablative elements circumferentially disposed about the cylindrical support structure respectively adjacent the plurality of electrodes;
an electrode configured for being deployed in the blood vessel of the patient at a location axially remote from the plurality of electrodes;
stimulation output circuitry;
monitoring circuitry;
an ablation source; and
a controller/processor configured for performing at least one of a first process and a second process,
wherein the first process comprises prompting the stimulation output circuitry to sequentially activate the plurality of electrodes to evoke at least one compound action potential in a nerve associated with the blood vessel, prompting the monitoring circuitry to activate the axially remote electrode in response to the activation of each of the plurality of electrodes to sense the at least one evoked compound action potential, and identifying one of the plurality of electrodes based on the at least one sensed compound action potential;
wherein the second process comprises prompting the stimulation output circuitry to active the axially remote electrode to evoke at least one compound action potential in the nerve associated with the blood vessel, prompting the monitoring circuitry to sequentially activate the plurality of electrodes in response to the activation of the axially remote electrode to sense the at least one evoked compound action potential, and identifying the one electrode based on the at least one sensed compound action potential,
wherein the controller/processor is configured for prompting the ablation source to delivering ablation energy to the ablative element adjacent the identified electrode.

2. The system of claim 1, wherein the cylindrical support structure comprises a resilient skeletal spring structure for urging the plurality of electrodes and the plurality of ablative elements against an inner wall of the blood vessel.

3. The system of claim 2, wherein the cylindrical support structure comprises an electrically insulative material for preventing electrical energy from being radially conveyed inward from the cylindrical support structure.

4. The system of claim 1, wherein the cylindrical support structure comprises one of a stent and a balloon.

5. The system of claim 1, wherein the cylindrical support structure carries the electrode.

6. The system of claim 1, wherein the electrode is a ring electrode.

7. The system of claim 1, wherein the plurality of ablative elements comprises the plurality of electrodes.

8. The system of claim 1, wherein the at least one evoked compound action potential comprises a plurality of evoked compound action potentials to increase the signal-to-noise ratio of the sensed evoked compound action potentials.

9. The system of claim 1, wherein the at least one the first process and the second process comprises the first process.

10. The system of claim 1, wherein the at least one of the first process and the second process comprises the second process.

11. The system of claim 1, wherein the ablation source comprises one of a thermal ablation source and a cryoablation source.

12. The system of claim 1, wherein the controller/processor is configured for automatically performing the at least one of the first process and the second process.

13. A method of treating a medical condition of a patient, comprising:
delivering electrical stimulation energy to a stimulation site on the wall of a blood vessel, thereby evoking at least one compound action potential in a nerve branch associated with the blood vessel;
sensing the at least one evoked compound action potential at a sensing site on the wall of the blood vessel;
identifying a circumferential location of the nerve branch as being adjacent one of the stimulation site and the sensing site based on the at least one sensed compound action potential; and
delivering ablation energy to an ablation site on the wall of the blood vessel adjacent the circumferential location of the nerve branch, thereby ablating the nerve branch and treating the medical condition.

14. The method of claim 13, further comprising:
disposing a stimulating electrode in the blood vessel at the stimulation site, wherein the electrical stimulation energy is delivered by the stimulating electrode; and
disposing a sensing electrode in the blood vessel at the sensing site, wherein the at least one evoked compound action potential is sensed by the sensing electrode.

15. The method of claim 13, wherein the one of the stimulation site and the sensing site is the stimulation site.

16. The method of claim 15, wherein the stimulation site and sensing site are axially remote from each other, the method further comprising:
disposing a plurality of stimulation electrodes in the blood vessel respectively at a plurality of circumferential sites in axial alignment with the stimulation site;
disposing a sensing electrode in the blood vessel at the sensing site;
sequentially activating the stimulation electrodes, wherein the at least one compound action potential is evoked by the activation of one of the stimulation electrodes;
activating the sensing electrode in response to the activation of each of the stimulation electrodes to sense the at least one evoked compound action potential; and
identifying the circumferential site at which the one stimulation electrode is located as the stimulation site.

17. The method of claim 16, further comprising:
disposing a plurality of ablative elements in the blood vessel respectively adjacent the stimulation electrodes; and
selecting the ablative element adjacent the one stimulation electrode to convey the ablation energy to the ablation site.

18. The method of claim 17, further comprising disposing a cylindrical support structure in the blood vessel in axial alignment with the stimulation site, wherein the stimulation electrodes and ablative elements are carried by the cylindrical support structure.

19. The method of claim 13, wherein the one of the stimulation site and the sensing site is the sensing site.

20. The method of claim 19, wherein the stimulation site and sensing site are axially remote from each other, the method further comprising:
disposing a plurality of sensing electrodes in the blood vessel respectively at a plurality of circumferential sites in axial alignment with the sensing site;
disposing a stimulation electrode in the blood vessel at the stimulation site;
activating the stimulation electrode to evoke the at least one compound action potential;
sequentially activating the sensing electrodes in response to the activation of the stimulation electrode, wherein the at least one evoked compound action potential is sensed by the activation of one of the sensing electrodes; and
identifying the circumferential site at which the one sensing electrode is located as the sensing site.

21. The method of claim 20, further comprising:
disposing a plurality of ablative elements in the blood vessel respectively adjacent the sensing electrodes; and
selecting the ablative element adjacent the one sensing electrode to convey the ablation energy to the ablation site.

22. The method of claim 21, further comprising disposing a cylindrical support structure in the blood vessel in axial alignment with the sensing site, wherein the sensing electrodes and ablative elements are carried by the cylindrical support structure.

23. The method of claim 13, wherein the ablation energy is one of thermal ablation energy and cryoablation energy.

24. The method of claim 13, wherein the at least one evoked compound action potential comprises a plurality of evoked compound action potentials to increase signal-to-noise ratio of the sensed evoked compound action potentials.

25. The method of claim 13, wherein the medical condition is hypertension, the blood vessel is a renal artery, and the ablation of the nerve branch decreases the blood pressure of the patient, thereby treating the hypertension.
